(19) United States
(12) Patent Application Publication
(10) Pub. No.: US 2015/0105668 A1
(43) Pub. Date: Apr. 16, 2015

(54) Endoscopic, Exoscopic Or Microscopic Apparatus For Fluorescence Diagnosis

(71) Applicant: Karl Storz GmbH & Co. KG, Tuttlingen (DE)

(72) Inventors: Andre Ehrhardt, Wurmlingen (DE); Gerd Beck, Wurmlingen (DE); Bernhard Gloeggler, Tuttlingen (DE); Klaus-Martin Irion, Emmingen-Liptingen (DE); Thomas Hinding, Aldingen (DE); Uwe Martin, Spaichingen (DE)

(21) Appl. No.: 14/514,933

(22) Filed: Oct. 15, 2014

(30) Foreign Application Priority Data
Oct. 15, 2013 (DE) 102013111368.0

(51) Int. Cl. A61B 5/00; A61B 1/00; G01N 21/64

(52) U.S. Cl. CPC A61B 5/0071; G01N 21/6486; A61B 1/00163; G01N 2201/0612; G01N 2201/062
USPC 600/476

(57) ABSTRACT

An endoscopic, exoscopic or microscopic apparatus for fluorescence diagnosis comprises a light source designed to emit light in a first spectral range and light in a second spectral range in a fluorescence mode The second spectral range is at least partly separate from the first spectral range. The light source has at least one first semiconductor illuminant which emits the light in the first spectral range in the fluorescence mode. The light source has at least one second semiconductor illuminant which emits the light in the second spectral range in the fluorescence mode. The apparatus comprises an open-loop or closed-loop control device, which keeps constant a preset ratio of a first intensity of the light in the first spectral range and a second intensity of the light in the second spectral range.

### CROSS REFERENCE TO RELATED APPLICATION

This application claims priority of German patent application No. 10 2013 111 368.0 filed on Oct. 15, 2013. The entire content of this priority application is incorporated herein by reference.

### BACKGROUND OF THE INVENTION

The invention generally relates to the field of fluorescence diagnosis. More specifically, the invention relates to an endoscopic, exoscopic or microscopic apparatus for fluorescence diagnosis, comprising a light source designed to emit light in a first spectral range and light in a second spectral range in a fluorescence mode, wherein the second spectral range is at least partly separate from the first spectral range:

An apparatus according to the invention for fluorescence diagnosis of the type mentioned in the introduction is preferably used for medical diagnosis purposes, but can also be used for technical diagnosis purposes in industrial or scientific applications.

In the context of medical fluorescence diagnosis, an apparatus of the type mentioned in the introduction is used for assessing the state of biological tissue, for example generally for tissue differentiation, in particular for tumor identification, but also for identifying blood circulation and vitality. With an apparatus for fluorescence diagnosis of the type mentioned in the introduction, the fluorescence diagnosis can be carried out in vivo, in particular.

In the medical field, fluorescence diagnosis has developed into a promising alternative or supplementation in the identification and treatment of neoplastic changes. Fluorescence diagnosis is based on the interaction of light having a suitable wavelength with a fluorescent substance present in the tissue area to be examined. In this case, a fluorescent substance can be a fluorescent dye previously introduced into the tissue area to be examined, for example by administration of the fluorescent substance itself or a precursor thereof to the patient to be examined. However, a fluorescent substance can also be a substance already present in the target region, a tissue-specific substance, for example, which is excited to autofluorescence by light in a suitable spectral range. The present invention can encompass both cases. The following explanations will assume the case that the fluorescent substance is a fluorescent dye introduced exogenously (from outside) into the tissue area to be examined.

In the case of the tumor-selective substances, a crucial role is currently accorded to 5-aminolevulinic acid (5-ALA) or its hexyl ester (5-ALAHE, trade name Hexvix®). It is an endogenous substance and the initial product of intracellular hambiosynthesis. After a number of reaction steps, endogenous porphyrins, primarily protoporphyrin IX (PPIX), are synthesized intracellularly. The latter is the crucial fluorochrome which is detected during the fluorescence-diagnostic examination. This is successful, however, only if 5-ALA is supplied to the afflicted organs exogenously in a sufficient concentration.

In present-day fluorescence diagnosis apparatuses, a xenon lamp is used as light source, and couples fluorescence excitation light into an endoscope via a flexible light transmission system. A camera system adapted for fluorescence diagnosis is used for video-technological documentation.

In the case of the conventional fluorescence diagnosis systems having a xenon lamp-based light source, an illumination filter is used in the illumination beam path and an observation filter is used in the observation beam path. The spectral transmission properties of the illumination filter and of the observation filter together determine the intensity of the backscattered short-wave excitation light perceived by the observer, said excitation light being blue in the case of PPIX. For an optimum contrast between fluorescent and non-fluorescent tissue, the reflected blue light passing to the detection stage must not be too intensive, since otherwise it is superimposed on the fluorescence, red fluorescence in the case of PPIX, to an excessively great extent. On the other hand, a certain minimum admixture of reflected blue light, the so-called blue tongue, is desirable in order to improve the diagnostics. It is known that backscattered light and fluorescent light should have approximately comparable intensity in order to obtain a good color contrast.

Without restricting the generality, within the meaning above, the first spectral range is the primary spectral range of the excitation of the fluorescence, and the second spectral range is a secondary spectral range (for example the blue tongue), light in the secondary spectral range being admixed to the fluorescence image.

One disadvantage of a xenon lamp-based light source is the relatively short lifetime of xenon lamps. Manufacturers of fluorescence diagnosis systems therefore recommend replacing the xenon lamp or the lamp module after a certain, relatively low number of operating hours. The disadvantage of such conventional fluorescence diagnosis systems therefore consists firstly in the additional expenditure in respect of costs caused by replacing the xenon lamp, and the outage times caused by the maintenance or replacement.

By contrast, semiconductor illuminants such as light-emitting diodes, which illuminants in the present application should be understood to encompass both inorganic and organic semiconductor illuminants, such as light-emitting diodes, laser diodes, superluminescence diodes and the like, have a significantly longer lifetime, as a result of which the abovementioned disadvantages can be avoided. Semiconductor illuminants nowadays also have a sufficient radiation power of several watts.

The document DE 10 2008 018 637 A1 proposes using a laser diode, light-emitting diode or superluminescence diode for the light source for fluorescence excitation.

DE 102 52 313 A1 discloses a fluorescence diagnosis apparatus comprising an excitation light source arrangement for emitting light in a spectral range of the excitation of a fluorescent dye and a further light source arrangement for emitting light in a further spectral range, which does not encompass the fluorescence spectrum and the excitation spectrum. In this case, an intensity of the further light source arrangement can be changed relative to an intensity of the excitation light source arrangement. The change in the intensity of the further light source arrangement is realized there by the introduction of different illumination filters into the illumination beam path, by illumination filters having adjustable transmission, and the like. Furthermore, an optimum relation between the maximum intensities in the two spectral ranges is stored in a computer, which relation was determined beforehand such that a user can perceive fluorescent regions and adjacent non-fluorescent regions in the fluorescence image simultaneously with good contrast, without the fluorescent radiation being swamped out. The computer drives a motor for rotating a filter wheel in such a way that the relation of the maximum intensities in the two spectral ranges substantially corresponds to the optimum relation. In the case of this apparatus, the user himself/herself coordinates the optimum relation of the maximum intensity of the further light source, which is then stored in the computer. However, the coordination of the optimum intensity ratio by the user entails the risk of a false positive and, in particular, false negative diagnosis being made.

The light source used there, which forms both the excitation light source and the further light source, is a broadband light source as is conventional, for example a halogen lamp, with the disadvantages described above.

While semiconductor illuminants have a long lifetime in comparison with xenon lamp-based light sources, the optical properties of semiconductor illuminants, e.g. the central wavelength, the full width at half maximum and the radiation power, vary within specific limits as a result of the production process. Furthermore, the optical properties and the lifetime of semiconductor illuminants are influenced by operating parameters, such as current intensity, voltage and temperature, for example. The optical properties can change (generally) reversibly during operation or else (generally) irreversibly over the course of the lifetime. When such light sources are used, this can have the effect that the color contrast changes undesirably during a session or over the course of time, as a result of which a reliable diagnosis is not ensured.

### SUMMARY OF THE INVENTION

It is an object of developing an apparatus of the type mentioned in the introduction to the effect that the color contrast required for a reliable diagnosis in the fluorescence image remains constant, specifically during a diagnosis session and also over the lifetime of the light source.

According to an aspect, an endoscopic, exoscopic or microscopic apparatus for performing fluorescence diagnosis is provided, comprising a light source having at least one first semiconductor illuminant to emit, in a fluorescence mode, light in a first spectral range, and at least one second semiconductor illuminant to emit, in the fluorescence mode, light in a second spectral range at least partly separate from the first spectral range, and a control device, which keeps constant a preset ratio of a first intensity of light in the first spectral range and a second intensity of light in the second spectral range.

The apparatus according to the invention for fluorescence diagnosis thus comprises semiconductor illuminants for emitting light in the excitation spectral range and also for emitting light in the secondary spectral range (for example for the blue tongue). As a result, firstly, the disadvantage of a comparatively short lifetime of conventional light sources of fluorescence diagnosis apparatuses comprising xenon or halogen lamp-based light sources is eliminated. The technical problem of the drifting of the optical properties of semiconductor illuminants is eliminated by an open-loop or closed-loop control device, which keeps constant a preset ratio of the light intensities in the first spectral range and in the second spectral range. In this case, the preset ratio of the abovementioned light intensities is not user-determined, but rather preferably determined clinically beforehand and stored in the fluorescence diagnosis apparatus, in particular in the open-loop or closed-loop control device thereof. The open-loop or closed-loop control device of the fluorescence diagnosis apparatus according to the invention now ensures that the preset intensity ratio is maintained both in the context of a diagnosis session and over the lifetime of the light source, i.e. the lifetime of the semiconductor illuminants, i.e. also independently of operating parameters such as current intensity, voltage and temperature and independently of the ageing of the semiconductor illuminants. The fluorescence diagnosis apparatus according to the invention thus always ensures a constant color contrast required for a reliable diagnosis in the fluorescence image.

The control device can be embodied as an open-loop control device or as a closed-loop control device. The open-loop or closed-loop control device can be embodied such that it controls the application of current and/or voltage to at least one of the semiconductor illuminants by open-loop or closed-loop control. It can also bring about open-loop or closed-loop control of the temperature of at least one of the semiconductor illuminants by means of cooling/heating in order to keep the preset intensity ratio constant.

The open-loop or closed-loop control device can be designed, in particular, to monitor the preset ratio of the first and second intensities for changes and, in the event of detected changes, to reset the actual ratio to the preset ratio. This last can be effected by virtue of the open-loop or closed-loop control device controlling the current intensity and/or voltage with which at least one of the semiconductor illuminants is operated by open-loop or closed-loop control.

The advantage of monitoring the preset intensity ratio is primarily that individual properties of the individual semiconductor illuminants which can differ from semiconductor illuminant to semiconductor illuminant can always be taken into account in the open-loop or closed-loop control, in particular individual differences in the semiconductor illuminants which arise only or primarily in the course of the ageing of the semiconductor illuminants.

In one preferred configuration of the measure mentioned above, the open-loop or closed-loop control device has a measuring device for measuring the intensities of the light emitted by the first semiconductor illuminant and the at least one second semiconductor illuminant.

This configuration of monitoring the preset intensity ratio can advantageously be realized in a simple manner; in particular, the measuring device used can be a photodiode which reacts to changes in brightness with a change in the current-voltage characteristic curve of the photodiode.

The apparatus according to the invention, as in conventional fluorescence diagnosis apparatuses, is preferably also equipped with an image acquisition device for acquiring a fluorescence image. In this case, it is preferred if the open-loop or closed-loop control device evaluates image signals of the image acquisition device in order to monitor the preset ratio for changes.

In this configuration, the basis of keeping the preset intensity ratio constant is advantageously the fluorescence image or the contrast thereof itself, wherein changes in the preset intensity ratio which are manifested in a change in the color contrast in the fluorescence image are acquired by the image acquisition device and used by the open-loop or closed-loop control device instantaneously for up-dating the actual intensity ratio to the preset intensity ratio.

As an alternative to monitoring the actual ratio for deviations with respect to the preset intensity ratio, a typical temporal profile of changes in the intensities of the light emitted by the first semiconductor illuminant and the at least one second semiconductor illuminant can be stored in the open-loop or closed-loop control device, wherein the open-loop or closed-loop control device calculates correction values on the basis of the typical temporal profile of the changes in order to keep the preset ratio constant.

This measure is based on the fact that a constant color contrast presupposes mutually coordinated current intensities of the semiconductor illuminant that emits the excitation light and the semiconductor illuminant that emits the secondary spectral range. The strengths of current of the semiconductor illuminants that are required for this purpose can be determined experimentally in the laboratory and/or in a clinical evaluation. These determined values are subsequently stored in the fluorescence diagnosis apparatus, for example in a memory module that can be arranged in the excitation light source. In this configuration of the apparatus, the open-loop or closed-loop control device is at least able to compensate for the preset intensity ratio drifting on the basis of empirical values, wherein keeping the preset intensity ratio constant is possibly subject to a tolerance. In this case, only open-loop control of the current intensity of at least one of the semiconductor illuminants takes place, but no closed-loop control. Nevertheless, this configuration has the advantage of a simpler construction and a lower complexity, since it is not necessary to monitor the actual intensity ratio.

Preferably, the open-loop or closed-loop control device is integrated into the light source.

The advantage here is a compact design of the fluorescence diagnosis apparatus.

The at least one first semiconductor illuminant and/or the at least one second semiconductor illuminant are/is a light-emitting diode (inorganic (LED) or organic (OLED)), which should also be understood to encompass a superluminescence diode, a laser diode, a light-emitting diode array or a laser diode array.

Preferably, the light source has at least one, preferably a plurality of further semiconductor illuminants which generate white light in a white light mode.

In this regard, the light source can additionally have a white light-emitting diode or a light-emitting diode array comprising red, green and blue LEDs which generate white light in combination with one another.

The apparatus according to the invention can preferably be switched between the fluorescence mode and the white light mode, wherein the white light mode serves for orienting the user in the observed area. In connection with the measures mentioned above it is preferred if the second semiconductor illuminant is an individual light-emitting diode that is active only with the first semiconductor illuminant in the fluorescence mode, while the second semiconductor illuminant is active with the further semiconductor illuminants for generating white light in the white light mode.

The second semiconductor illuminant is accordingly activated together with the first semiconductor illuminant in the fluorescence mode, wherein the second semiconductor illuminant then emits light in the second spectral range (secondary spectral range, for example for the blue tongue) for optimally imparting color contrast in the fluorescence image, while the second semiconductor illuminant is activated in combination with the further semiconductor illuminants for generating white light in the white light mode. In the case of fluorescence diagnosis by means of PPIX, the second semiconductor illuminant emits for the purpose of generating the blue tongue in the blue spectral range.

Preferably, the apparatus furthermore comprises a switching controller for switching between the fluorescence mode and the white light mode, wherein the switching controller for switching between the fluorescence mode and the white light mode switches the at least one first semiconductor illuminant, the at least one second semiconductor illuminant and the at least one further semiconductor illuminant.

While the auto-shutter of the image recording chip is used for switching between the fluorescence mode and the white light mode in conventional fluorescence diagnosis apparatuses, the measure mentioned above has the advantage that an autoshutter can be dispensed with.

In a further preferred configuration, a plurality of different preset ratios of the first and second intensities are stored in a memory, and it is possible to selectively switch between said ratios, wherein the open-loop or closed-loop control device keeps constant the preset ratio respectively selected.

As already mentioned above, it is generally necessary to determine the optimum color contrast, i.e. the optimum admixture of light in a secondary spectral range to the excitation, for example of blue light, in a clinical evaluation. The optimum color contrast can then turn out to be different in different applications (e.g. different organs, different diagnostic tasks, . . . ), i.e. there can be different preset intensity ratios for different organs. The measure mentioned above advantageously takes account of that. In this case, too, the open-loop or closed-loop control device keeps constant the respective preset intensity ratio.

It is likewise preferred if the light source has a plurality of second semiconductor illuminants which each generate light in different preset second spectral ranges, wherein it is possible to selectively switch between the different second spectral ranges.

This configuration has the advantage, on the one hand, that the fluorescence diagnosis apparatus can be designed for different fluorescent dyes, since it can generate light in different secondary spectral ranges. However, in the case of one and the same fluorescent dye, too, this measure has the advantage that light from different secondary spectral ranges can be admixed to the fluorescence image in order to obtain an optimum colour contrast for the diagnosis.

In a further preferred configuration, a beam combiner element is arranged between the at least one first semiconductor illuminant and the at least one second semiconductor illuminant.

In this case, it is advantageous that the first semiconductor illuminant and the at least one second semiconductor illuminant need not be arranged on a common optical axis, which would lead to spatial conflicts, rather the two semiconductor illuminants can be arranged with intersecting emission axes. The beam combiner element can be, for example, a plane plate having a reflective front side and a rear side that is transmissive to light in the relevant spectral range.

Furthermore, at least one optical element for collimation, which is preferably aspherized, is disposed downstream between the at least one semiconductor illuminant and the at least one second semiconductor illuminant.

A focusing optical unit has the advantage that the light emitted in an unfocused manner by the semiconductor illuminants can be focused for coupling into an optical waveguide connected to an endoscope, exoscope or a microscope, such that intensity losses when the light is coupled into the optical waveguide can be kept as small as possible.

In a further preferred configuration, the at least one second spectral range in which the at least one second semiconductor illuminant generates light is narrowband, and, in the case of the use of the fluorescence diagnosis apparatus with the fluorescent dye PPIX, the second semiconductor illuminant generates light preferably having a peak wavelength in the range of approximately 400 nm to approximately 500 nm, preferably of approximately 450 nm.

The narrowband nature of the second semiconductor illuminant for generating the secondary spectral range (the blue tongue in the case of PPIX) has the advantage that with skilful matching between the intensities of the secondary spectral range and of the primary spectral range, a red fluorescence appears with a different color contrast in the fluorescence image if the fluorescence is intensive, which indicates malignant tissue, in comparison with if the fluorescence is weak, i.e. no specific finding is present.

In a further preferred configuration, the preset ratio of first intensity and second intensity can be altered continuously or discretely during operation.

It was mentioned above that the user should not have a free choice of the intensity ratio, since this entails the risk of a false positive and in particular false negative diagnosis being made. However, the above measure has the advantage, for example during the training of physicians, that altered or additional values can also be predefined in non-diagnostic situations. As a result, during training it is possible to demonstrate how a change in the preset ratio of the intensities in the primary and secondary spectral ranges can have an effect on the color contrast and thus the diagnosis. This selection option can be useful even for experienced specialists in the field of fluorescence diagnosis.

The user can select a different value for the intensity ratio between primary and secondary spectral ranges in comparison with the preset intensity ratio, wherein the selected intensity ratio can deviate for example by a factor of between 1.05 and 100, preferably 1.1 and 20, relative to the preset ratio or ratios. In this case, it can furthermore be provided that it is possible to select between different fixedly preset intensity ratios, that intensity ratios which are different from those fixedly preset can be selected, or that the preset intensity ratio can be switched for a limited period of time.

Further advantages and features are evident from the following description and the accompanying drawing.

It goes without saying that the features mentioned above and those yet to be explained below can be used not only in the combination respectively indicated, but also in other combinations or by themselves, without departing from the scope of the present invention.

### BRIEF DESCRIPTION OF THE DRAWINGS

Exemplary embodiments of the invention are illustrated in the drawing and are described in greater detail hereinafter with reference to the drawing, in which:

FIG. 1 shows an endoscopic apparatus for fluorescence diagnosis in a block diagram;

FIG. 2 shows one exemplary embodiment of a light source of the apparatus in FIG. 1;

FIG. 3 shows a further exemplary embodiment of a light source of the apparatus in FIG. 1;

FIG. 4 shows a diagram for illustrating the effect of an admixture of light in a secondary spectral range to the primary spectral range of the excitation in a fluorescence image; and

FIG. 5 shows a flow chart of closed-loop control by which a preset intensity ratio of light in the primary spectral range to light in the secondary spectral range is kept constant.

### DETAILED DESCRIPTION OF PREFERRED EXEMPLARY EMBODIMENTS

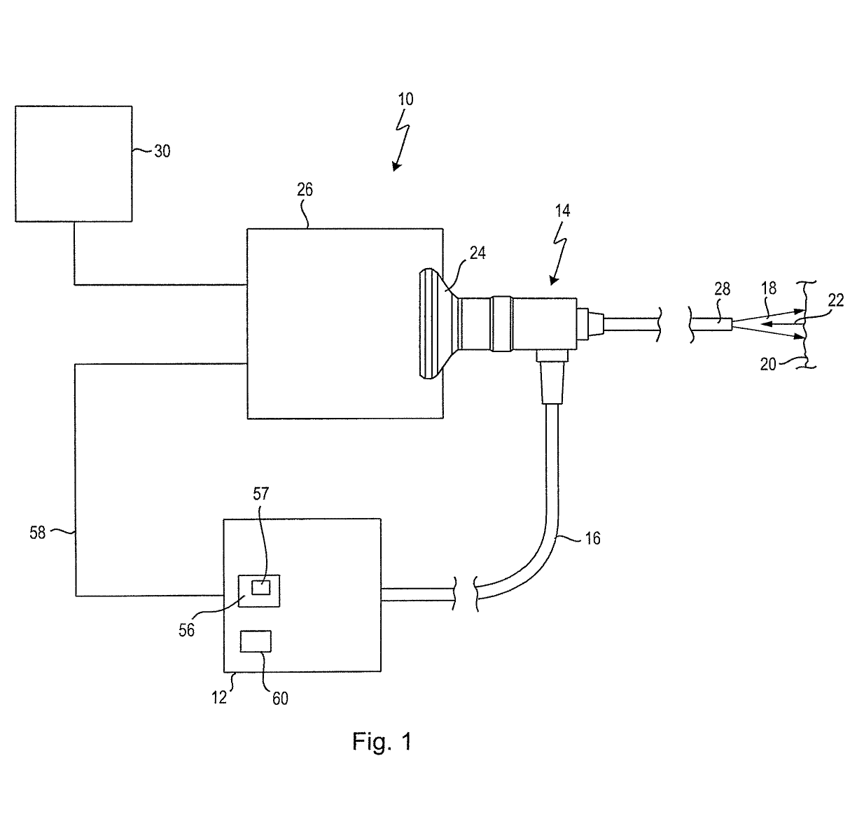

FIG. 1 shows an apparatus for fluorescence diagnosis, this apparatus being provided with the general reference sign 10. In the exemplary embodiment shown, the apparatus 10 is an endoscopic fluorescence diagnosis apparatus.

The apparatus 10 comprises a light source 12, which emits light in a first spectral range and light in a second spectral range in a fluorescence mode, as will be described later. In this case, the second spectral range is at least partly separate from the first spectral range.

The apparatus 10 furthermore comprises an endoscope 14, which is connected to the light source 12 via an optical waveguide in the form of a fibre-optic cable 16.

Light emitted by the light source 12 is fed into the endoscope 14 via the fibre-optic cable 16 and is directed by said endoscope, as indicated by a light cone 18, onto an area 20 to be observed, for example a tissue area in a human or animal body.

The light in the first spectral range generated by the light source 12 serves for exciting fluorescence of a fluorescent dye concentrated in the area 20. Such a fluorescent dye is PPIX, for example, as was described above. Accordingly, the first spectral range mentioned above is the spectral range of the fluorescence excitation of the fluorescent dye. This first spectral range is also called primary spectral range hereinafter.

The fluorescent dye present in the area 20 is excited to fluorescence by the light in the primary spectral range. The fluorescent light emitted by the fluorescent dye, this light being indicated by an arrow 22, is received by the endoscope 14 and passed through the optical system (not shown) of the endoscope, which can be formed by lenses or a fibre-optic unit, to an eyepiece 24, to which an image acquisition device 26, preferably a camera, is connected.

It goes without saying that the image acquisition device 26 can also be integrated into the endoscope 14, nowadays miniaturized cameras being available which can even be integrated into the tip 28 of the endoscope 14.

Furthermore, it goes without saying that the apparatus 10 can comprise a microscope or an exoscope instead of the endoscope 14.

The image acquisition device 26 is connected to an image reproduction device 30, for example a monitor.

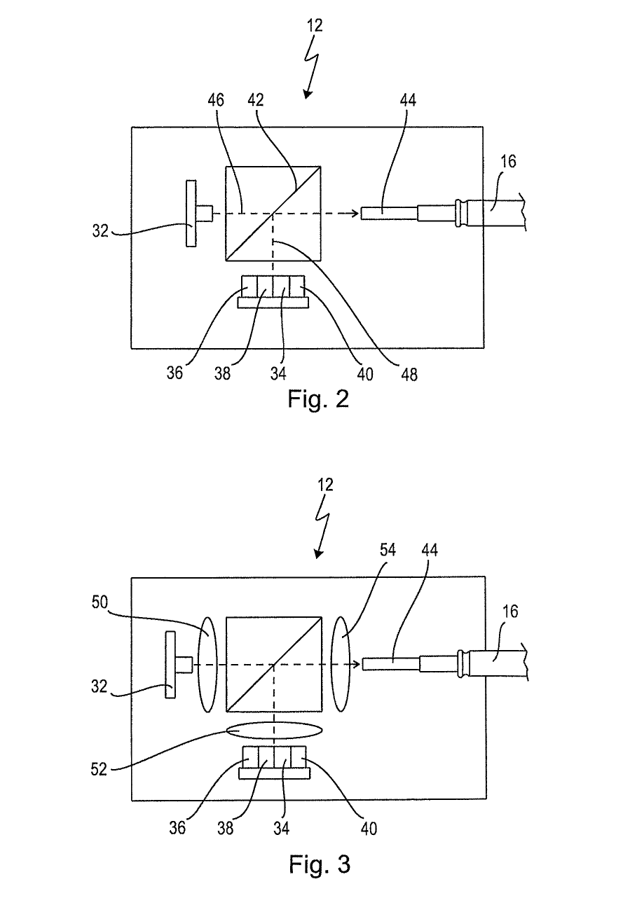

Before further details of the apparatus 10 are described, exemplary embodiments of the light source 12 will firstly be described with reference to FIGS. 2 and 3.

The light source 12 has a first semiconductor illuminant 32, which emits light in the first spectral range, i.e. the primary spectral range of the fluorescence excitation.

In the example of the fluorescent dye PPIX, the semiconductor illuminant 32 emits light in the ultraviolet in a narrowband range having a peak wavelength of approximately 405 nm.

The first semiconductor illuminant 32 can be embodied as a light-emitting diode (inorganic or organic), as a laser diode, as a light-emitting diode array or as a laser diode array.

The light source 12 has at least one second semiconductor illuminant 34, and also further semiconductor illuminants 36, 38 and 40. The semiconductor illuminants 34, 36, 38 and 40 form an array and are embodied in each case as one or a plurality of light-emitting diodes (organic or inorganic) or as laser diodes.

The second semiconductor illuminant 34 emits light in a second spectral range, which is at least partly separate from the first spectral range, in which the first semiconductor illuminant 32 emits. The second spectral range is also designated as the secondary spectral range hereinafter. The second semiconductor illuminant 34 serves to emit light in the secondary spectral range which is reflected from the area 20 by scattering or reflection and is admixed to the fluorescent light in the fluorescence mode in order to obtain an optimum color contrast in the fluorescence image for the diagnosis.

In the present exemplary embodiment, the second semiconductor illuminant 34 emits light in the blue (so-called blue tongue) in a narrowband spectral range having a peak wavelength of approximately 450 nm. Consequently, the light emitted by the second semiconductor illuminant 34 has a somewhat longer wavelength than the light emitted by the first semiconductor illuminant 32.

The further semiconductor illuminants 36, 38 and 40 are, for example, individual light-emitting diodes (organic or inorganic), wherein for example the semiconductor illuminant 36 is a light-emitting diode which emits in the green spectral range, the semiconductor illuminant 38 is a light-emitting diode which emits in the red spectral range, and the semiconductor illuminant 40 is a light-emitting diode which emits white light.

In a white light mode of the apparatus 10, the semiconductor illuminants 34, 36, 38 and 40 generate white light.

Only the second semiconductor illuminant 34 together with the first semiconductor illuminant 32 is activated in the fluorescence mode, and the second semiconductor illuminant 34 together with the further semiconductor illuminants 36, 38 and/or 40 is activated for generating white light in the white light mode.

The light source 12 furthermore has a beam combiner element 42, which is transmissive on its side facing the first semiconductor illuminant 32 and is reflective on the side facing the semiconductor illuminants 34, 36, 38 and 40.

The beam combiner element 42 combines the light emitted by the semiconductor illuminants 32 and also 34, 36, 38 and 40 for joint coupling into one end of one or a plurality of optical fibres 44 of the fibre-optic cable 16 for forwarding to the endoscope 14.

On account of the beam combiner element 42, the individual semiconductor illuminants 32, 34, 36, 38 and 40 can be arranged relative to one another such that their emission axes 46 and 48 are at an angle, in particular a right angle, with respect to one another.

FIG. 3 shows an exemplary embodiment of the light source 12 which is modified in comparison with the exemplary embodiment in FIG. 2 and which differs from the exemplary embodiment in FIG. 2 in that an optical unit 50 is disposed downstream of the first semiconductor illuminant 32, an optical unit 52 is disposed downstream of the second semiconductor illuminant 34 and the further semiconductor illuminants 36, 38 and 40, and an optical unit 54 is disposed downstream of the beam combiner element 42. The optical units 50, 52 and 54 can be aspherized. Particularly if the semiconductor illuminants 32 and respectively 34, 36, 38 and 40 are embodied as arrays of light-emitting diodes or laser diodes, the optical units 50, 52 and 54 have the advantage that they collimate the emitted light to a narrow cross section and the light can thus be coupled into the fibre or fibres 44 of the fibre-optic cable 16 in a manner free of losses.

In the fluorescence mode of the apparatus 10, for an optimum color contrast for a reliable diagnosis it is essential that the ratio of the intensity of the fluorescence excitation light emitted by the first semiconductor illuminant 32 and the intensity of the light in the secondary spectral range emitted by the second semiconductor illuminant 34 has a specific value or at least lies in a specific narrow range of values. The optimum intensity ratio between the light in the first spectral range (primary spectral range) and second spectral range (secondary spectral range) is optimally determined in clinical studies. The optimum intensity ratio thus determined (or the optimum range of intensity ratios thus determined) is stored as preset intensity ratio in a memory, for example in the light source 12.

During the operation of the light source 12 in a diagnosis session or over the lifetime of the light source 12, i.e. of the semiconductor illuminants 32, 34, the optical properties of the semiconductor illuminants 32 and 34 can change, in particular change differently with respect to one another, which has the consequence that the abovementioned intensity ratio of light in the primary spectral range and light in the secondary spectral range likewise changes. Such a change results in an alteration of the color contrast in the fluorescence image, which makes it more difficult to interpret the fluorescence image during the diagnosis of malignant or benign tissue and can even entail the risk of false diagnoses.

Against this background, the apparatus 10 comprises an open-loop or closed-loop control device 56, which keeps constant the abovementioned preset intensity ratio of the light in the primary spectral range and the light in the secondary spectral range.

In this case, the preset intensity ratio can be kept constant by the respective supply of current or voltage to the semiconductor illuminant 32 and the semiconductor illuminant 34 being controlled by open-loop or closed-loop control. Since, in the case of semiconductor illuminants, the temperature also has an influence on the emitted intensity, provision can also be made for the open-loop or closed-loop control device 56 to control the temperature of the semiconductor illuminants 32, 34 by open-loop or closed-loop control in interaction with cooling/heating (not shown).

The open-loop or closed-loop control device 56 is integrated into the light source 12.

The open-loop or closed-loop control device 56 can be embodied merely as an open-loop control device, but it is preferably embodied as a closed-loop control device.

Particularly in the case of the configuration as a closed-loop control device, the open-loop or closed-loop control device 56 is designed to monitor the preset ratio of the intensity of the light in the first spectral range and the intensity of the light in the second spectral range for changes and, in the event of detected changes, to reset or update the present or actual ratio of the intensities to the preset ratio. This can be realized, as will be described later, by virtue of the open-loop or closed-loop control device 56 having a measuring device 57 for measuring the intensity emitted by the semiconductor illuminant 32 and for measuring the intensity emitted by the semiconductor illuminant 34.

However, the image acquisition device 26, which acquires the fluorescence image and the image signals of which are fed to the open-loop or closed-loop control device 56 via a connection 58, can also be used for monitoring the preset intensity ratio. The open-loop or closed-loop control device 56 then evaluates the image signals communicated by the image acquisition device 26 in short time intervals in order to check whether the preset intensity ratio changes. In the case of a change in the preset intensity ratio, the open-loop or closed-loop control device 56 correspondingly drives the semiconductor illuminants 32 and/or 34 independently of one another in order, by means of adapted energization thereof, to reestablish the preset intensity ratio.

As an alternative to the above-described configuration of the open-loop or closed-loop control device 56 as a closed-loop control device, it can also be embodied merely as an open-loop control device. In this case, a previously determined typical temporal profile of changes in the intensities emitted by the semiconductor illuminants 32, 34 is stood, for example, in a memory module of the open-loop or closed-loop control device 56. Such a typical temporal profile of changes in the emitted intensities can be determined, for example, by the temporal profile of the emitted intensities of the first and of the second semiconductor illuminant 32 and 34, respectively, being determined beforehand. The typical temporal profile stored in the open-loop or closed-loop control device 56, for example, is then used for calculating correction values with which the open-loop or closed-loop control device 56 correspondingly drives the first semiconductor illuminant 32 and/or the second semiconductor illuminant 34 in order to keep the preset intensity ratio constant.

In the case of the apparatus 10, the switching between the fluorescence mode and the white light mode is effected by the corresponding switching of the at least one first semiconductor illuminant 32, of the at least one second semiconductor illuminant 34 and of the further semiconductor illuminants 36, 38 and 40. For this purpose, a switching controller 60 is present, which can be integrated into the light source 12.

A plurality of different preset ratios of the first intensity in the primary spectral range and the second intensity in the secondary spectral range can be stored in a memory, for example in a memory module present in the open-loop or closed-loop control device 56, and the user can selectively switch between said ratios, wherein the open-loop or closed-loop control device 56 then keeps constant the preset ratio respectively selected.

In a modification of the exemplary embodiment of the light sources 12 in FIGS. 2 and 3, the light source 12 can have not just one second semiconductor illuminant 34, but a plurality of second semiconductor illuminants 34, for which purpose the semiconductor illuminants 36, 38 or 40 can also be used, which each generate light in different predetermined second spectral ranges, i.e. secondary spectral ranges, such that in each case light from one or a plurality of said second semiconductor illuminants can selectively be switched on in order to admix light in a different secondary spectral range to the fluorescent light, wherein the invention provides that the intensity ratio must remain constant, however.

Finally, it can also be provided that the preset intensity ratio between primary spectral range and secondary spectral range can be altered continuously or discretely during the operation of the apparatus 10, the altered intensity ratio then being kept constant by the open-loop or closed-loop control device 56. The optional change in the preset ratio of the intensities in the primary spectral range and secondary spectral range can be limited temporally, in particular, while a switch is made to the preset intensity ratio again after a predetermined time.

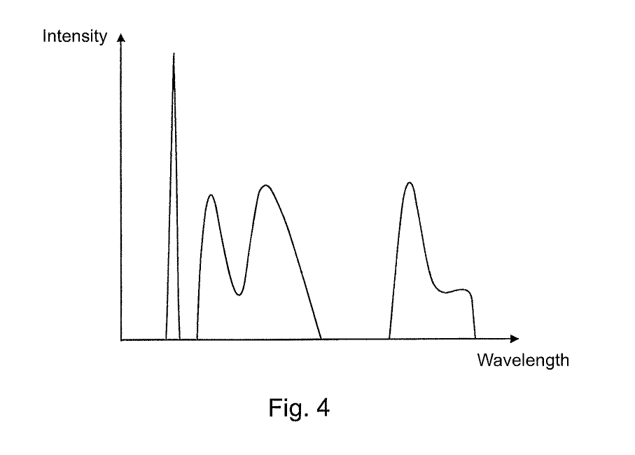

FIG. 4 schematically shows a diagram of how the secondary spectral range (middle curve) has an effect on the fluorescence image. The narrowband nature of the emission of the second semiconductor illuminant 34 for generating the secondary spectral range (the blue tongue in the case of PPIX) has the advantage that with skilful matching between the intensities of the secondary spectral range and of the primary spectral range, a red fluorescence appears with a different color contrast in the fluorescence image if the fluorescence is intensive, which indicates malignant tissue, in comparison with if the fluorescence is weak, i.e. no specific finding is present.

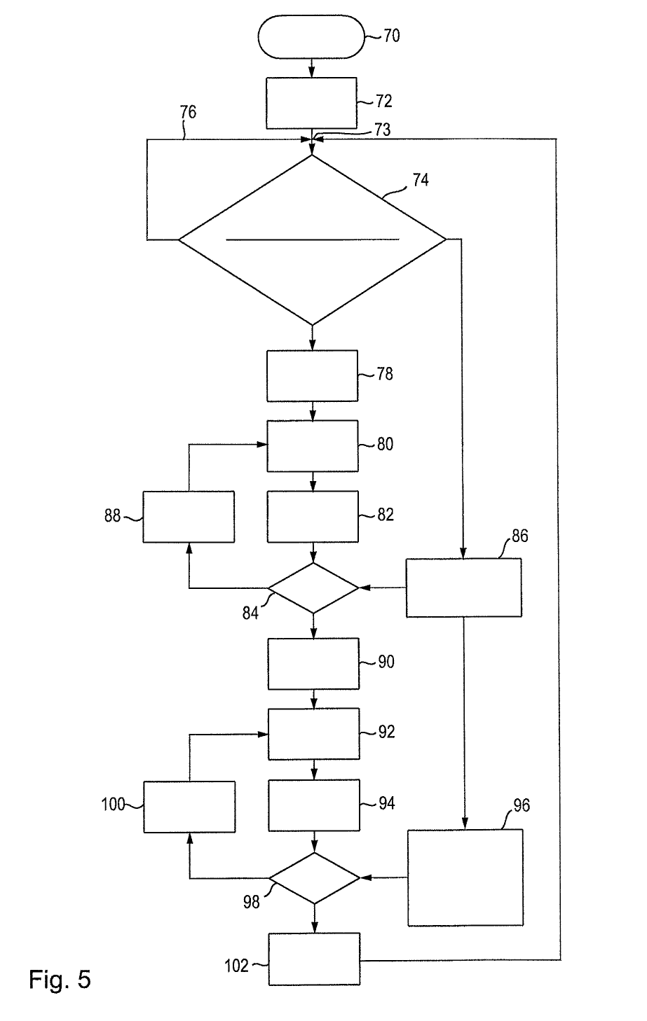

FIG. 5 shows a flow chart of how a preset intensity ratio of the intensity in the primary spectral range and the intensity in the secondary spectral range can be kept constant. In this case, the user has no influence on the sequence of the closed-loop control described below. In this case, the closed-loop control including monitoring of the preset intensity ratio proceeds in a very short time interval (for example milliseconds), such that the closed-loop control does not adversely affect the user during the use of the apparatus 10.

As a result of the switching-on 70 of the light source 12, the first semiconductor illuminant 32 and the second semiconductor illuminant 34 are switched on in the fluorescence mode (reference sign 72). The starting point 73 of the closed-loop control is reached after the semiconductor illuminants 32 and 34 have been switched on. The semiconductor illuminants 32 and 34 emit light in the primary spectral range and secondary spectral range with a present or actual intensity ratio. At 74, a check is then made to determine whether the present or actual intensity ratio deviates from the preset intensity ratio. If this is not the case, there is a return to the starting point again via 76.

The checking of the present or actual intensity ratio at 74 is carried out at the beginning, i.e. after the switching-on of the light source 70, and after arbitrary time intervals and also in the case of a manual change of the brightness of the light source 12.

If the check at 74 reveals a deviation or change of the present or actual ratio from the preset intensity ratio, the following further sequence of the closed-loop control by the open-loop or closed-loop control device 56 takes place.

At 78, firstly the second semiconductor illuminant 34, which emits light in the secondary spectral range, is switched off, such that at 80 only the first semiconductor illuminant 32, which emits light in the primary spectral range, is switched on. At 82, the intensity of the light emitted by the first semiconductor illuminant 32 is measured by means of the measuring device 57. The measurement can be effected for example by means of an optical sensor, for example a photodiode of the measuring device 57. The measured value is subsequently compared with reference values applicable to the preset intensity ratio (reference sign 86) at 84. If deviations between the measured intensity and the stored reference values occur, the energization of the first semiconductor illuminant 32 is correspondingly adapted at 88.

If the comparison at 84 reveals that the intensity emitted by the first semiconductor illuminant 32 is correct, the first semiconductor illuminant 32 is switched off at 90, while only the second semiconductor illuminant 34 is then switched on at 92. The suitable intensity value of the emission of the second semiconductor illuminant 34 is then determined in the subsequent course of the flow chart. This procedure takes place analogously to the determination of the correct intensity value of the first semiconductor illuminant 32, i.e. at 94 the intensity of the emission of the second semiconductor illuminant 34 is measured and compared with reference values (reference sign 96) for the preset intensity ratio and, if appropriate, with an additionally adjustable intensity offset for the second semiconductor illuminant 34 at 98 and, if necessary, at 100 the energization of the second semiconductor illuminant 34 is correspondingly changed. If the comparison at 98 reveals that the intensity emitted by the second semiconductor illuminant 34 is correct, at 102 the first semiconductor illuminant 32 is switched on, and the closed-loop control returns to the starting point 73 again, and the monitoring and closed-loop control of the preset intensity ratio starts anew.

What is claimed is:

1. An apparatus, comprising
a light source having
at least one first semiconductor illuminant to emit, in a fluorescence mode, light in a first spectral range, and
at least one second semiconductor illuminant to emit, in the fluorescence mode, light in a second spectral range at least partly separate from the first spectral range, and
a control device, which keeps constant a preset ratio of a first intensity of light in the first spectral range and a second intensity of light in the second spectral range,
the apparatus being an apparatus for performing fluorescence diagnosis.

2. The apparatus of claim 1, wherein the control device is an open-loop control device.

3. The apparatus of claim 1, wherein the control device is a closed-loop control device.

4. The apparatus of claim 1, wherein the control device is designed to monitor the preset ratio of the first and second intensities of light for changes and, in the event of detected changes, to reset an actual ratio to the preset ratio.

5. The apparatus of claim 4, wherein the control device has a measuring device for measuring the first and second intensities of light emitted by the first semiconductor illuminant and the at least one second semiconductor illuminant.

6. The apparatus of claim 4, furthermore comprising an image acquisition device for acquiring a fluorescence image, wherein the control device evaluates image signals of the image acquisition device in order to monitor the preset ratio for changes.

7. The apparatus of claim 1, wherein a typical temporal profile of changes in the first and second intensities of light emitted by the first semiconductor illuminant and the at least one second semiconductor illuminant is stored in the control device, and the control device calculates correction values on the basis of the typical temporal profile of changes in order to keep the preset ratio constant.

8. The apparatus of claim 1, wherein the control device is integrated into the light source.

9. The apparatus of claim 1, wherein the at least one first semiconductor illuminant is selected from a group consisting of a light-emitting diode, a laser diode, a light-emitting diode array, a laser diode array.

10. The apparatus of claim 1, wherein the at least one second semiconductor illuminant is selected from a group consisting of a light-emitting diode, a laser diode, a light-emitting diode array, a laser diode array.

11. The apparatus of claim 1, wherein the light source has at least one further semiconductor illuminant switched on in a white-light mode.

12. The apparatus of claim 11, wherein the second semiconductor illuminant is an individual light-emitting diode that is active only with the first semiconductor illuminant in the fluorescence mode, and wherein the second semiconductor illuminant is active with the at least one further semiconductor illuminant for generating white light in the white-light mode.

13. The apparatus of claim 11, further comprising a switching controller for switching between the fluorescence mode and the white light mode, wherein the switching controller for switching between the fluorescence mode and the white light mode switches the at least one first semiconductor illuminant, the at least one second semiconductor illuminant and the at least one further semiconductor illuminant.

14. The apparatus of claim 1, further comprising a memory, wherein a plurality of different preset ratios of the first and second intensities are stored in the memory for individual selection of any one of the preset ratios, and wherein the control device keeps constant the preset ratio respectively selected.

15. The apparatus of claim 1, wherein the light source has a plurality of second semiconductor illuminants which each generate light in different preset second spectral ranges, wherein the light source is switchable between the different second spectral ranges.

16. The apparatus of claim 1, further comprising a beam combiner arranged between the at least one first semiconductor illuminant and the at least one second semiconductor illuminant.

17. The apparatus of claim 1, further comprising at least one optical element for collimation disposed downstream of the at least one first semiconductor illuminant and the at least one second semiconductor illuminant.

18. The apparatus of claim 17, wherein the at least one optical element for collimation has at least one aspherized surface.

19. The apparatus of claim 1, wherein the at least one second spectral range in which the at least one second semiconductor illuminant generates light is narrowband.

20. The apparatus of claim 19, wherein the at least one second spectral range has a peak wavelength in the range of approximately 400 nm to approximately 500 nm.

21. The apparatus of claim 19, wherein the at least one second spectral range has a peak wavelength of approximately 450 nm.

22. The apparatus of claim 1, wherein the preset ratio of the first intensity and the second intensity can be altered during operation.

23. The apparatus of claim 1, wherein the apparatus comprises an instrument selected from a group consisting of an endoscope, exoscope, microscope.
